(19) United States
(12) Patent Application Publication
(10) Pub. No.: US 2019/0365630 A1
(43) Pub. Date: Dec. 5, 2019

(54) ANTIBACTERIAL PARTICLES AND METHODS

(71) Applicant: The Procter & Gamble Company, Cincinnati, OH (US)

(72) Inventor: David Frederick Swaile, Cincinnati, OH (US)

(21) Appl. No.: 16/424,571

(22) Filed: May 29, 2019

Related U.S. Application Data
(60) Provisional application No. 62/678,330, filed on May 31, 2018.

(51) Int. Cl. A61K 8/81; A61K 8/02; A61K 8/04; A61K 8/26; A61Q 15/00; A61K 8/20

(52) U.S. Cl. CPC A61K 8/817; A61K 8/0229; A61K 8/046; A61K 8/26; A61Q 15/00; A61K 2800/34; A61K 2800/31; A61K 2800/5426; A61K 2800/412; A61K 2800/74; A61K 8/20

(57) ABSTRACT

Compositions and methods include an antibacterial particle comprising a cationically charged polymer and a salt.

### CROSS REFERENCE TO RELATED APPLICATION

This application claims the benefit of U.S. Provisional Application No. 62/678,330, filed May 31, 2018, the substance of which is incorporated herein by reference.

### FIELD

Deodorant and antiperspirant compositions comprising an antibacterial particle comprising a cationically charged polymer and a salt and methods thereof.

### BACKGROUND

One of the main functions of a deodorant or antiperspirant product is to control unpleasant body odor. At least some body odor is the result of microorganisms on the skin which break down sweat to produce the smell that is associated with body odor. Thus, there is a need for deodorant and antiperspirant compositions that neutralize body odor by targeting the microorganisms that create the body odor. While many antibacterials are known to formulators, not just any antibacterial is easily incorporated into any deodorant or antiperspirant product or any product form. There may be processing difficulties or compatibility issues for the antibacterial with the other materials in the product. For example, polyvinylamine polymers are particularly good at controlling bacteria known to cause body odor. However, while polyvinylamine is known as an antibacterial in aqueous solution, drying the polymer results in a hygroscopic solid or semi-solid that cannot be easily ground to a cosmetically acceptable powder. Further, the polymer by itself is too hygroscopic to be incorporated into antiperspirant or deodorant products without picking up moisture that can cause interaction with antiperspirant active particles. Thus, there is a need for a way to include materials that have good antibacterial effect successfully into antiperspirant and deodorant products, particularly anhydrous sticks or sprays.

### SUMMARY

An antibacterial particle comprising a cationically charged polymer and a salt.

### BRIEF DESCRIPTION OF THE DRAWINGS

FIG. 1 is a graph showing the consumer-perceived efficacy of a product comprising an inventive particle in comparison to a control product.

FIG. 2 is a graph showing the results of an odor reduction clinical test for a product comprising an inventive particle in comparison to a control product.

### DETAILED DESCRIPTION

The components and/or steps, including those which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “anhydrous” as used herein means substantially free of added or free water. From a formulation standpoint, this means that the anhydrous antiperspirant stick compositions of the present invention contain less than about 1%, and more specifically zero percent, by weight of free or added water, other than the water of hydration typically associated with the particulate antiperspirant active prior to formulation. In other words, water is not added as a product ingredient, but because some other components, such as the antiperspirant active, have water, the final product may have at most 5% water.

The term “ambient conditions” as used herein refers to surrounding conditions under about one atmosphere of pressure, at about 50% relative humidity, and at about 25° C., unless otherwise specified. All values, amounts, and measurements described herein are obtained under ambient conditions unless otherwise specified.

The term “polarity” as used herein is defined by the Hansen Solubility Parameter for solubility.

“Substantially free of” refers to about 2% or less, about 1% or less, or about 0.1% or less of a stated ingredient. “Free of” refers to no detectable amount of the stated ingredient or thing. The term “volatile” as used herein refers to those materials that have a measurable vapor pressure at 25° C. Such vapor pressures typically range from about 0.01 millimeters of Mercury (mm Hg) to about 6 mmHg, more typically from about 0.02 mmHg to about 1.5 mmHg; and have an average boiling point at one (1) atmosphere of pressure of less than about 250° C., more typically less than about 235° C. Conversely, the term “non-volatile” refers to those materials that are not “volatile” as defined herein.

“Deodorant composition” as used herein refers to a composition that is applied to at least a portion of the body, which is used to combat body odor.

“Leave-on” as used herein refers to a composition that is designed to be applied to at least a portion of the body and then left on that portion of the body.

### I. Antibacterial Particle

### Cationically Charged Polymers

The particles of the present invention can provide an antiperspirant or deodorant product an odor reduction benefit, even without the inclusion of a fragrance. The odor reduction may be provided by the particle's antibacterial effect. The antibacterial particles of the present invention may comprise a highly cationically charged polymer and a water soluble salt. Certain cationally charged polymers are known to have antibacterial effects, but by themselves cannot be dried to an easily friable particle. For example, primary and secondary amines, and more particularly polyvinyl amines, when dried in an attempt to incorporate into an anhydrous deodorant or antiperspirant product, often form a soft, sticky semi-solid polymer. This hygroscopic gel cannot easily be ground into a cosmetically acceptable powder.

The present inventors have discovered that adding a salt to the cationically charged polymer and then co-spray drying the mixture can create a friable particle that can be dispersed in an anhydrous product. This allows the polymer antibacterial properties to be delivered directly to the skin. These friable polymer particles are also compatible with antiperspirant actives. In addition, the dilution effect of co-spray drying the polymer with salt increases the number of antibacterial particles versus the polymer alone if they were added at equal concentration. Thus, use of these particles increases the antibacterial coverage of a dose of the product.

As mentioned, an exemplary cationically charged polymer of the present invention may be primary and secondary amines, or more specifically, polyvinylamines.

Polyvinylamines (PVams) are water soluble and are formed from a polyamine polymer having primary amine groups. A PVam is a linear polymer with pendent, primary amine groups directly linked to the main chain of alternating carbons. PVams are manufactured from hydrolysis of poly(N-vinylformamide) (PVNF) which results in the conversion of formamide units to amino groups as described by the following formula (IIa):

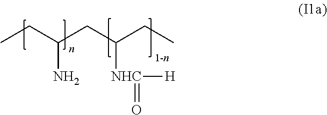

where n is a number from 0.1 to 0.99 depending on the degree of hydrolysis. For instance, in 95% hydrolyzed PVam polymer, n will be 0.95 while 5% of the polymer will have formamide units.

PVams may be partially hydrolyzed meaning that 1% to 99%, 30% to 99%, 50% to 99%, 70% to 99%, 80% to 99%, 85% to 99%, 90% to 99%, 95% to 99%, 97% to 99%, or about 99% of the PVam is hydrolyzed. It has been found that a high degree of hydrolysis of PVam increases the resulting polymer's ability to mitigate the odors.

PVams that can be hydrolyzed may have an average molecular weight (MW) of 5,000 to 350,000. Suitable hydrolyzed PVams are commercially available from BASF. Some examples include Lupamin® 9095, 9030, 9010, 5095, and 1595.

Other cationically charged polymers may include, but are not limited to, polyvinyl amine polyvinyl formide copolymer, polyquaterium polymers, polylysine, and combinations thereof.

The cationically charged polymers of the present invention may be highly cationically charged. The cationically charged polymers of the present invention may have a minimum molecular weight of 2000, and have at least about 30% of monomers with a cationic charge. In some embodiments, the polymers may have a minimum molecular weight of 2000, of 4000, 6000, 8000, or 10,000, and at least about 40%, about 50%, about 70%, or about 95% of monomers with a cationic charge.

Tables 1 and 2 show the minimum inhibition concentration of polyvinyl formamide against two known bacteria associated with body odor, Corynebacterium mucifaciens and Staphylococcus epidermidis, respectively. The data shows that good bacterial control occurs when the polyvinyl formamide has at least about 30% of the monomers with a positive charge, and when its molecular weight is 10,000 and above.

TABLE 1
Minimum inhibition concentration for Corynebacterium mucifaciens

| Average | | | | | | | |
|---|---|---|---|---|---|---|---|
| Molecular | | | | | | | |
| weight of | | | | | | | |
| polyvinyl | Percent of monomers that are hydrolyzed to amine | | | | | | |
| formamide | 0 | 10 | 20 | 30 | 50 | 70 | 95 |
| 10,00 | | | | | | | 3.1 ppm |
| 45,000 | >100 | >100 | | 6.25 ppm | 12.5 ppm | 25 ppm | 50 |
| | ppm | ppm | | | | | ppm |
| 340,000 | >100 | >100 | | 12.5 ppm | 50 ppm | | 12.5 |
| | ppm | ppm | | | | | ppm |

TABLE 2
Minimum inhibition concentration for Staphylococcus epidermidis

| Average | | | | | | | |
|---|---|---|---|---|---|---|---|
| Molecular | | | | | | | |
| weight of | | | | | | | |
| polyvinyl | Percent of monomers that are hydrolyzed to amine | | | | | | |
| formamide | 0 | 10 | 20 | 30 | 50 | 70 | 95 |
| 10,00 | | | | | | | 3.1 ppm |
| 45,000 | >100 | >100 | | 12.5 ppm | 12.5 ppm | 25 ppm | 50 |
| | ppm | ppm | | | | | ppm |
| 340,000 | >100 | >100 | | 25 ppm | 50 ppm | | 12.5 |
| | ppm | ppm | | | | | ppm |

The pH of the cationically charged polymer (measured in a 5% solution of polymer in water) may be less than about 7, in some embodiments, less than about 5, and in other embodiments less than about 4. The lower pH values, due to increased cationic charge, are believed to have less interaction with antiperspirant actives, and also give a better yield in the spray drying process. This undesirable interaction is best controlled by varying the pH of the polymer/salt feed solution to the dryer.

The deodorant or antiperspirant composition may comprise from about 0.01% to about 20%, by weight of the composition, of the cationically charged polymer. The composition may also comprise from about 0.5%, about 1.0%, about 1.5%, about 2%, about 3%, about 5%, to about 2.0%, about 3.0%, about 5%, about 10%, about 15%, about 20%, or any combination thereof, by weight of the composition, of the cationically charged polymer. While these polymers often come in solution form, the amounts of polymer are by active percentage, not percentage of raw material.

### Salts

The present invention may combine the cationically charged polymer with a salt. A wide variety of salts may be used, including water-soluble, inorganic salts. Suitable salts may have at least about 5% solubility in water, in some cases at least about 10% and in still other cases at least about 20% solubility. While not being bound by theory, it is thought that this allows the particle to dissolve on skin and makes the polymer available for odor control. If the salt is not water soluble enough it may not release the polymer to act as an antibacterial agent. However if the salt is too hygroscopic, there may be drawbacks. In some embodiments, the salt used may have a hygroscopicity such that it absorbs less than about 40% of its weight at 80% humidity, and in some cases, less than about 20% of its weight at 80% humidity. The polymer dried by itself, without a salt, is often too hygroscopic to be incorporated into deodorant and antiperspirant products without picking up moisture that could cause interaction with antiperspirant active particles, or that could cause the product to clump during shipping, making the product difficult to later disperse. But mixing the polymer with a salt and then drying allows the hygroscopicity to be controlled, thereby preventing negative interactions and allowing convenient shipping of the material.

Table 3 below shows the weight % absorbed at different humidities for the salt sodium chloride and then also for the antibacterial particle (polymer plus salt) made in Example 4 and used in Examples 1-3. The data was generated by a DVS (Dynamic Vapor Sorption) Intrinsic Instrument as detailed below in the Test Methods. Table 3 demonstrates that both the salt alone and the polymer plus salt absorb less than 40% of their weight at 80% humidity.

TABLE 3

| | % Difference | % Difference | % Difference |
|---|---|---|---|
| | @ 50% RH | @ 80% RH | @ 85% RH |
| Sodium Chloride | 2.14 | 2.94 | 6.27 |
| Antibacterial Particle | 3.62 | 9.41 | 14.2 |
| from Example 4 | | | |

Salts that may be used in the present invention may include, but are not limited to, sodium chloride, potassium chloride, sodium potassium aluminum sulfate (alum), aluminum sulfate, aluminum chlorohydrate, aluminum zirconium salts, lithium chloride, calcium chloride, magnesium chloride, and combinations thereof.

The deodorant and antiperspirant compositions may comprise from about 1% to about 15%, in some embodiments from about 2% to about 12%, and in some embodiments from about 5% to about 10%, by weight of the composition, of the antibacterial particle (polymer plus salt).

### II. Other Deodorant and Antiperspirant Components

The deodorant and antiperspirant composition may also include additional ingredients like, for example, emollients, propellants, solubilizers, chelants, anti-oxidants, fragrances, encapsulates, structurants, thickeners, gelling agents, deodorant and antiperspirant actives, other actives, buffering agent, preservatives, dyes, and combinations thereof, etc.

### Emollients

Deodorant and antiperspirant compositions can comprise an emollient system including at least one emollient, but it could also be a combination of emollients. Suitable emollients are often liquid under ambient conditions. Depending on the type of product form desired, concentrations of the emollient(s) in the deodorant compositions can range from about 1% to about 95%, from about 5% to about 95%, from about 15% to about 75%, from about 1% to about 10%, from about 15% to about 45%, or from about 1% to about 30%, by weight of the deodorant composition.

Emollients suitable for use in the compositions include, but are not limited to, propylene glycol, polypropylene glycol (like dipropylene glycol, tripropylene glycol, etc.), diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, alkyl ethers of polyhydric and monohydric alcohols, volatile silicone emollients such as cyclopentasiloxane, non volatile silicone emollients such as dimethicone, mineral oils, polydecenes, petrolatum, and combinations thereof. One example of a suitable emollient comprises PPG-15 stearyl ether. Other examples of suitable emollients include dipropylene glycol and propylene glycol. Further examples of suitable emollients may include mineral oil; PPG-14 butyl ether; isopropyl myristate; petrolatum; butyl stearate; cetyl octanoate; butyl myristate; myristyl myristate; C12-15 alkylbenzoate (e.g., Finsolv™); octyldodecanol; isostearyl isostearate; octododecyl benzoate; isostearyl lactate; isostearyl palmitate; isobutyl stearate; dimethicone, and any mixtures thereof.

### Propellant

The compositions described herein may include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42%, or any combination thereof, by weight of the total fill of materials stored within the container.

### Antiperspirant Actives

The antiperspirant stick compositions of the present invention can comprise a particulate antiperspirant active suitable for application to human skin. The concentration of antiperspirant active in the composition should be sufficient to provide the desired perspiration wetness and odor control from the antiperspirant stick formulation selected.

The antiperspirant stick compositions of the present invention comprise an antiperspirant active at concentrations of from about 0.5% to about 60%, and more specifically from about 5% to about 35%, by weight of the composition. These weight percentages are calculated on an anhydrous metal salt basis exclusive of water and any complexing agents such as, for example, glycine, and glycine salts. The antiperspirant active as formulated in the composition can be in the form of dispersed particulate solids having an average particle size or equivalent diameter of less than about 100 microns, more specifically less than about 20 microns, and even more specifically less than about 10 microns.

The antiperspirant active for use in the anhydrous antiperspirant compositions of the present invention may include any compound, composition or other material having antiperspirant activity. More specifically, the antiperspirant actives may include any of the antibacterial discussed above, or may also include astringent metallic salts, especially inorganic and organic salts of aluminum, zirconium and zinc, as well as mixtures thereof. Even more specifically, the antiperspirant actives may include aluminum-containing and/or zirconium-containing salts or materials, such as, for example, aluminum halides, aluminum chlorohydrate, aluminum hydroxyhalides, zirconyl oxyhalides, zirconyl hydroxyhalides, and mixtures thereof.

Aluminum salts for use in the anhydrous antiperspirant stick compositions include those that conform to the formula:

Al₂(OH)ₐCl_{b}.xH₂O,

wherein a is from about 2 to about 5;

the sum of a and b is about 6;

x is from about 1 to about 6; and

a, b, and x may have non-integer values.

More specifically, aluminum chlorohydroxides referred to as “⅚ basic chlorohydroxide” may be used, wherein a=5, and “⅔ basic chlorohydroxide”, wherein a=4.

Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980, the disclosures of which are incorporated herein by reference for the purpose of describing processes for preparing aluminum salts.

Mixtures of aluminum salts are described in British Patent Specification 1,347,950, Shin et al., published Feb. 27, 1974, which description is also incorporated herein by reference.

Zirconium salts for use in the anhydrous antiperspirant stick compositions include those which conform to the formula:

ZrO(OH)₂₋ₐClₐ.xH₂O,

wherein a is from about 1.5 to about 1.87;

x is from about 1 to about 7; and

a and x may both have non-integer values.

These zirconium salts are described in Belgian Patent 825,146, Schmitz, issued Aug. 4, 1975, which description is incorporated herein by reference. Zirconium salts that additionally contain aluminum and glycine, commonly known as “ZAG complexes,” are believed to be especially beneficial. These ZAG complexes contain aluminum chlorohydroxide and zirconyl hydroxy chloride conforming to the above-described formulas. Such ZAG complexes are described in U.S. Pat. No. 3,792,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978, disclosures of which are incorporated herein by reference for the limited purpose of describing ZAG complexes.

Also suitable for use herein are enhanced efficacy aluminum-zirconium chlorohydrex-amino acid which typically has the empirical formula AlₙZr(OH)_{[3n+4-m(n+1)]}(Cl)_{[m(n+1)]}-AA_{q} where n is 2.0 to 10.0, preferably 3.0 to 8.0; m is about 0.48 to about 1.11 (which corresponds to M:Cl approximately equal to 2.1-0.9), preferably about 0.56 to about 0.83 (which corresponds to M:Cl approximately equal to 1.8-1.2); q is about 0.8 to about 4.0, preferably about 1.0 to 2.0; and AA is an amino acid such as glycine, alanine, valine, serine, leucine, isoleucine, β-alanine, cysteine, β-amino-n-butyric acid, or γ-amino-n-butyric acid, preferably glycine. These salts also generally have some water of hydration associated with them, typically on the order of 1 to 5 moles per mole of salt (typically, about 1% to about 16%, more typically about 4% to about 13% by weight). These salts are generally referred to as aluminum-zirconium trichlorohydrex or tetrachlorohydrex when the Al:Zr ratio is between 2 and 6 and as aluminum-zirconium pentachlorohydrex or octachlorohydrex when the Al:Zr ratio is between 6 and 10. The term “aluminum-zirconium chlorohydrex” is intended to embrace all of these forms. The preferred aluminum-zirconium salt is aluminum-zirconium chlorohydrex-glycine. Additional examples of suitable high efficacy antiperspirant actives can include Aluminum Zirconium Pentachlorohydrex Glycine, Aluminum Zirconium Octachlorohydrex Glycine, or a combination thereof. These high efficacy actives are more fully described in U.S. App. Pub. No. 2007/0003499 by Shen et al. filed Jun. 30, 2005.

Some embodiments may be aluminum-free, or substantially free of aluminum. In some embodiments, antibacterials may be selected from the group consisting of 2-Pyridinol-N-oxide (piroctone olamine), beryllium carbonate, magnesium carbonate, calcium carbonate, magnesium hydroxide, magnesium hydroxide and magnesium carbonate hydroxide, partially carbonated magnesium hydroxide, potassium carbonate, potassium bicarbonate, sodium carbonate, sodium sesquicarbonate, baking soda, hexamidine, zinc carbonate, thymol, polyvinyl formate, salycilic acid, niacinamide and combinations thereof.

### Deodorant Actives

Suitable optional deodorant actives may include any topical material that is known or otherwise effective in preventing or eliminating malodor associated with perspiration. Suitable deodorant actives may be selected from the group consisting of antibacterial agents (e.g., bacteriocides, fungicides), malodor-absorbing material, and combinations thereof. For example, antibacterial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

### Structurants

The antiperspirant and deodorant compositions of the present invention comprise a suitable concentration of a primary structurant to help provide the compositions with the desired viscosity, rheology, texture and/or product hardness, or to otherwise help suspend any dispersed solids or liquids within the composition.

The term “solid structurant” as used herein means any material known or otherwise effective in providing suspending, gelling, viscosifying, solidifying, and/or thickening properties to the composition or which otherwise provide structure to the final product form. These solid structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof.

The concentration and type of solid structurant selected for use in the deodorant and antiperspirant compositions will vary depending upon the desired product hardness, rheology, and/or other related product characteristics. For most structurants suitable for use herein, the total structurant concentration ranges from about 5% to about 35%, more typically from about 10% to about 30%, or from about 7% to about 20%, by weight of the composition.

Non-limiting examples of suitable primary structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; solid triglycerides; behenyl alcohol, or combinations thereof.

Other non-limiting examples of primary structurants suitable for use herein are described in U.S. Pat. No. 5,976,514 (Guskey et al.) and U.S. Pat. No. 5,891,424 (Bretzler et al.), the descriptions of which are incorporated herein by reference.

The antiperspirant composition can further comprise an additional structurant. The additional structurant may be present in an amount from 1% to about 10%, by weight of the composition. The additional structurant(s) will likely be present at an amount less than the primary structurant.

Non-limiting examples of suitable additional structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof.

Other non-limiting examples of additional structurants suitable for use herein are described in U.S. Pat. No. 5,976,514 (Guskey et al.) and U.S. Pat. No. 5,891,424 (Bretzler et al.).

### Solvent

The antiperspirant composition of the present invention comprises a solvent at concentrations ranging from about 20% to about 80%, and more specifically from about 30% to about 70%, by weight of the composition. The solvent can be a volatile silicone which may be cyclic or linear.

“Volatile silicone” as used herein refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976), which descriptions are incorporated herein by reference.

The volatile silicone can be a cyclic silicone having from 3 to 7, and more specifically from 5 to 6, silicon atoms, and still more specifically 5, like cyclopentasiloxane. These cyclic silicone materials will generally have viscosities of less than about 10 centistokes at 25° C. In some cases, the antiperspirant or deodorant product may be free from or substantially free of added cyclic silicone, meaning the only cyclic silicone present may be from other silicone raw materials that may contain low levels (less than 5%) of cyclic silicone. In other cases the antiperspirant or deodorant product may contain less than about 1% or less than about 5% cyclic silicone. In still other cases, the APDO product may contain about 10% to about 70% cyclic silicone.

Linear volatile silicone materials suitable for use in the antiperspirant compositions include those represented by the formula:

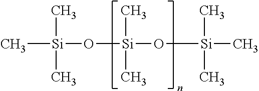

wherein n is from 1 to 7, and more specifically from 2 to 3. These linear silicone materials will generally have viscosities of less than about 5 centistokes at 25° C.

Specific examples of volatile silicone solvents suitable for use in the antiperspirant compositions include, but are not limited to, Cyclomethicone D-5; GE 7207 and GE 7158 (commercially available from General Electric Co.); Dow Corning 344; Dow Corning 345; Dow Corning 200; and DC1184 (commercially available from Dow Corning Corp.); and SWS-03314 (commercially available from SWS Silicones).

Non-Volatile Organic Fluids

Non-volatile organic fluids may be present, for example, in an amount of about 15% or less, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate, and isobutyl stearate.

### Perfumes and Fragrance Delivery

The compositions herein may include microcapsules. The microcapsules may be any kind of microcapsule disclosed herein or known in the art. The microcapsules may have a shell and a core material encapsulated by the shell. The core material of the microcapsules may include one or more fragrances. The shells of the microcapsules may be made from synthetic polymeric materials or naturally-occurring polymers. The microcapsules may be friable microcapsules. A friable microcapsule is configured to release its core material when its shell is ruptured. The rupture can be caused by forces applied to the shell during mechanical interactions. The microcapsules may have shells made from any material in any size, shape, and configuration known in the art. Some or all of the shells may include a polyacrylate material, such as a polyacrylate random copolymer. The microcapsules may also encapsulate one or more benefit agents. The benefit agent(s) include, but are not limited to, one or more of chromogens, dyes, cooling sensates, warming sensates, fragrances, oils, pigments, in any combination. When the benefit agent includes a fragrance, said fragrance may comprise from about 2% to about 80%, from about 20% to about 70%, from about 30% to about 60% of a perfume raw material with a ClogP greater than −0.5, or even from about 0.5 to about 4.5. The microcapsules may encapsulate an oil soluble material in addition to the benefit agent. The microcapsule may be spray-dried to form spray-dried microcapsules. The personal care compositions may also include a parent fragrance and one or more encapsulated fragrances that may or may not differ from the parent fragrance. Some fragrances may be considered to be volatile and other fragrances may be considered to be or non-volatile. Further types and processes regarding microcapsules are disclosed in U.S. Pat. No. 9,687,425.

The composition may also contain one or more other delivery systems for providing one or more benefit agents, in addition or in place of the microcapsules. The additional delivery system(s) may differ in kind from the microcapsules. For example, wherein the microcapsule are friable and encapsulate a fragrance, the additional delivery system may be an additional fragrance delivery system, such as a moisture-triggered fragrance delivery system. Non-limiting examples of moisture-triggered fragrance delivery systems include cyclic oligosaccaride, starch (or other polysaccharide material), or combinations thereof. Further details regarding suitable starches and cyclic oligosaccharide are disclosed in U.S. Pat. No. 9,687,425.

The compositions may include one or more fragrances. As used herein, “fragrance” is used to indicate any odoriferous material. Any fragrance that is cosmetically acceptable may be used in the antiperspirant and deodorant compositions. For example, the fragrance may be one that is a liquid at room temperature. Generally, the fragrance(s) may be present at a level from about 0.01% to about 40%, from about 0.1% to about 25%, from about 0.25% to about 20%, or from about 0.5% to about 15%, by weight of the personal care composition.

A wide variety of chemicals are known as fragrances, including aldehydes, ketones, and esters. More commonly, naturally occurring plant and animal oils and exudates comprising complex mixtures of various chemical components are known for use as fragrances. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release. The fragrances herein may be relatively simple in their chemical make-up, comprising a single chemical, or may comprise highly sophisticated complex mixtures of natural and synthetic chemical components, all chosen to provide any desired odor. Suitable fragrances are also disclosed in U.S. Pat. Nos. 9,687,425, 4,145,184, 4,209,417, 4,515,705, and 4,152,272.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

### Buffering Agent

The solution of polymer and salt may include a buffering agent which may be alkaline, acidic or neutral. The buffer may be used in the solution of polymer and salt for controlling and/or maintaining the desired pH. The solution of polymer and salt may have a pH from about 3 to about 10, from about 4 to about 9, from about 5 to about 8, from about 6 to about 7, or it may have a pH of about 6.5. One unique aspect of the polyvinyl amine polymers are their ability to maintain active nitrogen sites at high pH levels which can help enhance the antibacterial effect which comes, at least in part, from the nitrogen sites.

Suitable buffering agents include, for example, hydrochloric acid, sodium hydroxide, potassium hydroxide, citric acid, and combinations thereof.

The solution of polymer and salt may contain at least about 0%, alternatively at least about 0.001%, alternatively at least about 0.01%, by weight of the solution, of a buffering agent. The solution may also contain no more than about 1%, alternatively no more than about 0.75%, alternatively no more than about 0.5%, by weight of the solution, of a buffering agent.

### Water

The deodorant and antiperspirant compositions may be anhydrous.

The deodorant compositions herein may be in any suitable form, including, for example, solid sticks, roll-on deodorants, gel sticks, or body sprays.

### Other Compositions

The particles of the present invention may be incorporated into deodorant or antiperspirant compositions and products, but may also be incorporated into a variety of personal care compositions and products. For example, the particles of the present invention may be incorporated into compositions used for treating hair, such as bleaching, coloring, dyeing, conditioning, growing, removing, retarding growth, shampooing, styling. The particles of the present invention may be incorporated into compositions and products used for personal cleansing, for color cosmetics, for treating skin, including topically applied products and orally administered materials, or the particles may be incorporated into compositions and products used for treating nails, or for shaving.

### III. Method of Making

The antibacterial particles comprising a cationically charged polymer and a salt may be made as follows: First provide a solution of cationically charged polymer and add to it a salt in at least about five times in excess of the cationically charged polymer. Then dry both the polymer and salt to an anhydrous particle, and grind the particle to less than about 75 microns.

The particles may be from about 5 microns to about 50 microns. In some embodiments, such as stick deodorant and antiperspirant products, the particle size may be less than about 10 microns, or from about 1 to about 10 microns. In some embodiments, such as sprays, the particle size may be from about 20 microns to about 30 microns. For spray products, the minimum particle size may be 10 microns, wherein less than about 10% of the particles are less than about 10 microns.

As noted above, the dilution effect of co-spray drying the polymer with the salt increases the number of antibacterial particles compared to drying the polymer alone. In anhydrous deodorant and antiperspirant products, having a larger particle count can increase the antibacterial coverage of the underarm area. For example, co-spray drying polyvinylamine with sodium chloride can increase the polymer, to where a product with 7% powder particles delivers about 1% polymer. This roughly seven fold increase in volume of antibacterial particles increases the homogeneity of the particles within stick and soft solid products, maximizes the release of polymer from product forms with high level of water insoluble components (i.e. waxes in sticks), and increases the probability that the antibacterial particle will be deposited near a sweat gland or hair follicle where odor causing bacteria reside.

The deodorant and/or antiperspirant composition can be made in any suitable manner known in the art. For example, base spray and roll on chassis formulations can be prepared as pre-mixes (i.e. all of the ingredients except for the antibacterial particle are added to a vessel, stirred, and heated if necessary), and the antibacterial particle can be added to a pre-mix.

### IV. Methods of Use and Methods of Reducing Body Malodor

Deodorant and antiperspirant compositions can be used, for example, by rubbing the composition on the skin via an applicator or by spraying the composition on the skin. Application can be achieved by using a spray device, a roller, a pad, a nozzle, etc. A method of use could be, for example, applying to a user a leave-on deodorant composition comprising an effective amount of an antibacterial polymer comprised of a cationically charged polymer and a salt. Another method of use could include applying to a user a leave-on deodorant composition comprising an effective amount of an antibacterial particle and an antiperspirant active.

Also included herein are methods for reducing body malodor by reducing the population of bacteria in the underarm. A method for reducing body malodor can include, for example, applying a leave-on deodorant composition comprising an effective amount of an antibacterial particle comprising a cationically charged polymer and a salt to at least a portion of the underarm of a user.

Another method includes a method of reducing underarm bacteria, comprising: applying a leave-on deodorant composition comprising an antibacterial particle comprising a cationically charged polymer and a salt to at least a portion of the underarm of a user.

While some compositional components are listed in the methods section for illustration, the deodorant compositions in the methods can contain any combination of components as discussed above in the Deodorant and Antiperspirant Components section.

### V. Test Methods

A. In Vitro Inhibition of Odor Causing Bacteria—Minimal Inhibitory Concentration (MIC) Method (Tables 1 and 2):

Overnight cultures of clinically isolated strains of Corynebacteria mucifaciens or Staphylococcus epidermidis (obtained from the underarms of volunteer subjects) were created by using a single colony from a streak plate to inoculate 50 mL of Muller-Hinton+1% Tween-80 (MHT) broth. Cultures were incubated overnight ˜18-22 hrs in a shaking incubator (33° C., 200 rpm). A 1:1000 dilution of the respective culture was made in MHT broth and placed on ice. The 1:1000 inoculums were quantitated by plating onto MHT-agar to monitor the number of colony forming units (CFUs) added to the study plate(s). Plates were incubated 24 hrs (S. epidermidis) or 48 hrs (C. mucifaciens) at 33° C. Typically 105-106 CFUs (S. epidermidis) or 104-105 CFUs (C. mucifaciens) were found to be added to each assay well.

Test compositions were assayed in triplicate employing 1:2 serial dilutions using MHT broth for a volume of 100 μl per well in a 96-well flat bottom culture plate. Once study materials were dispensed, 100 μl per well of the appropriate 1:1000 bacterial culture was used to inoculate. The assay plates were placed onto a plate shaker (˜500 rpm) and incubated at 33° C. for 24-28 hrs. The MIC was determined by visualization. Briefly, the lowest dose that remains transparent after serial dilution was determined to be the MIC value (100% inhibition of bacteria growth) for a given study material. The greater the dilution required to see the onset of bacterial growth the more potent the antibacterial activity of the test material.

B. Self Assessed Body Odor Clinical Methods and Results

Individuals were subject to a 3-day washout period where no antiperspirant or deodorant was used and only soap and shampoo usage was permitted. Following the 3-day washout period a 10-day treatment study followed. For 10 consecutive days, the individual's underarm was limited to one treatment per day with the given tested trial product, and the individual was not permitted to use any antiperspirant or deodorant product. The dose of the tested trial product (e.g., leave on roll-on personal care composition) was limited to 500 mg/underarm.

For Assessment of Anti-Odor Efficacy: Subjects self assessed their underarm odor at baseline and twice/day (am & pm) throughout the clinical trial. For their individualized odor assessment, each subject removed their shirt, turned their head towards the underarm and sniffed. An odor score was assigned for each underarm using a 0-10 rating scale where 0 indicated no detectable odor and 10 represented the highest level of odor this subject had personally experienced on themselves. Underarm odor scores were designated as “12 hour” and “24 hour”; where “12 hour” scores represent odor measures taken in the evening, or about 12 hours after product use and “24 hour” scores represent odor measures taking the following morning, or about 24 hours after product use.

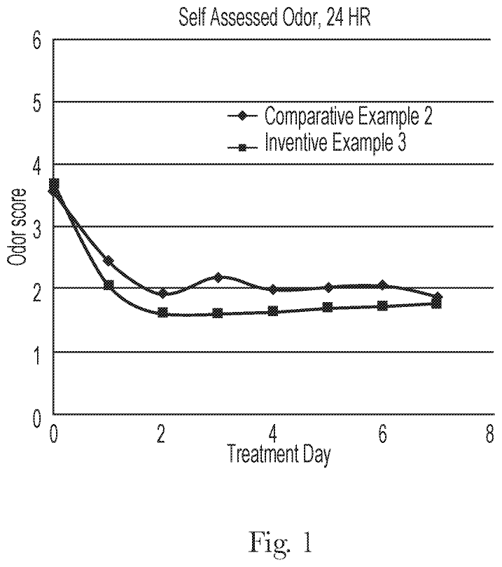

FIG. 1 shows the consumer-perceived efficacy of a product comprising an inventive particle (Inventive Example 3) in comparison to a control product (Comparative Example 2) not comprising an inventive particle, using the Self Assessed Body Odor Clinical Method described above. This demonstrates that consumers perceive a lower amount of body odor in the axilla treated with the inventive particle on days 1-6. The noticeable improvement in body odor was surprising, as both products contained 24% Aluminum Zirconium Trichlorohydrex-gly which is known in the art to provide significant odor protection via both wetness and bacterial control. Without being bound by theory, it is believed that the noticeable improvement at 24 hours after application is created by the inventive particle providing a more long-lasting bacterial control benefit than previously known material.

C. DVS Intrinsic Isotherm

The water sorption properties of solid materials are critical factors in determining their storage, stability, processing and application performance. Water sorption has traditionally been measured by storing materials in sealed jars containing saturated salt solutions that provide a range of relative humidity conditions. The materials are then removed and weighed once they reach equilibrium to determine water sorption/desorption.

The DVS (Dynamic Vapor Sorption) Intrinsic instrument, such as that used to generate the data of Table 3, allows sensitive, accurate, rapid, and automated determination of water sorption properties of solids. The DVS Intrinsic uses a sample size of a few mgs of the solid to be measured. The sample is placed on a sensitive balance and the sample chamber is sealed. The instrument is programmed to deliver a dynamic flow of moisture to the sample chamber. The difference in weight of the sample is measured to determine water sorption/desorption.

### Test Conditions:

Temperature: 25 C

˜60 mg sample

Humidity Conditions: 0% Humidity for 1 hour, then step up 10% humidity each hour until 100% humidity, then step down to 0% humidity for 1 hour.

### Plot % Mass Difference vs % Actual Relative Humidity

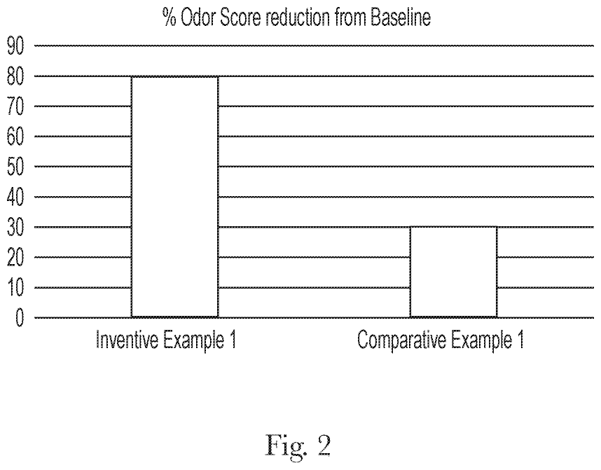

FIG. 2 shows the results of an odor reduction clinical test based on the ASTM E1207-14 method comparing example 1 with comparative example 1. In this test 18 panelists, who had not used underarm products for 9 days, were graded for underarm odor intensity 12 hours after a controlled wash to establish a baseline odor score. The example products were then applied to their underarms (bilateral, randomized by panelist) for 5 days. 12 hours after the last application, their underarm odor was graded again and a % reduction from baseline was calculated. As can be seen in the FIG. 2, the example containing the inventive particle provides a large statistically significant odor reduction increase as compared to the comparative example. As mentioned above, it is believed that this odor reduction is provided by a long lasting bacterial control effect on the skin.

### EXAMPLES

Inventive Examples 1-3, which are anhydrous sticks, are shown below in Table 4, along with Comparative Examples 1 and 2.

TABLE 4

| | Comparative | Inventive | Inventive | Comparative | Inventive |
|---|---|---|---|---|---|
| | Example 1 | Example 1 | Example 2 | Example 2 | Example 3 |
| Ingredient | A | B | C | D | E |
| Cyclopentasiloxane | 41.82 | 33.42 | 46.985 | 29.9425 | 27.9325 |
| Aluminum | | | 26.49 | 24.00 | 24.00 |
| Zirconium | | | | | |
| Trichlorohydrex-gly | | | | | |
| Stearyl Alcohol | 20.63 | 20.63 | | 14.00 | 11.90 |
| Mineral Oil | 12.00 | 12.00 | | 1.00 | 1.00 |
| Antibacterial | | 8.40 | 8.40 | | 7.69 |
| Particle ** | | | | | |
| Dimethicone | 7.50 | 7.50 | | | |
| Talc | 6.50 | 6.50 | | 3.00 | |
| Petrolatum | 4.50 | 4.50 | 3.00 | 3.00 | 3.00 |
| Hydrogentated | 3.75 | 3.75 | | 3.85 | 3.27 |
| Castor Oil | | | | | |
| PPG-14 Butyl | 3.00 | 3.00 | 0.50 | 6.50 | 6.50 |
| Ether | | | | | |
| Behenyl Alcohol | 0.30 | 0.30 | | 0.20 | 0.20 |
| Dimethicone 10 cst | | | 5.00 | | |
| Tribehenin | | | 4.50 | | |
| C18-36 Acid | | | 1.125 | | |
| Triglycerides and | | | | | |
| Tribehenin | | | | | |
| Datura BCD | | | 3.00 | 2.00 | 2.00 |
| Perfume | | | 1.00 | 0.0075 | 0.0075 |
| C12-15 Alkyl | | | | 9.50 | 9.50 |
| Benzoate | | | | | |
| Phenyltrimethicone | | | | 3.00 | 3.00 |

** Inventive Antibacterial Particle as made in Example 4.

### Example 4: Antibacterial Particle and Making of Antibacterial Particle

A batch of antibacterial particles was created using the following procedure. The following were added to a large container: 12.15 lbs. of water, 9.74 lbs. of Lupamin 1595 solution (available from BASF), and 4.87 lbs. of sodium chloride. The pH of this solution was then adjusted to 3 using 20 Baume HCL. The liquid mixture was then equilibrated for 24 hours. The equilibrated liquid was then spray dried using a Bowen Spray-Aire Model #BE-994 spray dryer. Spray dryer conditions were as follows: Inlet Temperature 180° C.±2° C., Outlet temperature 110° C.±2° C., and feed rate of solution 120-130 mL/mi. The dryer was fitted with an atomizer disc outlet. The resulting powder was then pulverized using a Hosokawa Alpine AFG, Model 100 pulverizer. Puliverizer conditions were as follows: classifier speed 6700-7350 rpm, mill pressure −1 to −3 inches of H₂O, system pressure 37-39 inches of H₂O, gas flow 50-52 SCFM, and grind pressure 70 psi.

TABLE 5
Key characteristics of the feed solution
and antibacterial powder in Example 4

| Feed solution | | Final Powder | |
|---|---|---|---|
| pH of feed solution | 2.98 | Yield unpulverized (lbs.) | 6.76 |
| Percent Chloride | 15.7 | Yield pulverized (lbs.) | 5.7 |
| | | % Yield | 84.3 |
| | | pH (15% in water, wt/wt) | 4.19 |
| | | Percent chloride | 51.2 |
| | | Percent Nitrogen | 3.06 |
| | | Particle size - D50 (microns) | 5.3 |

TABLE 6
Inventive Example 5 - Spray Example

| Ingredient | |
|---|---|
| Aluminum Chlorohydrate¹ | 26.37% |
| Antibacterial Particle² | 5% |
| Dimethicone³ | 38% |
| Isopropyl Myristate | 9% |
| Hydrophilic tapioca material⁴ | 7% |
| Stearalkonium Hectorite⁵ | 4.25% |
| Triethyl Citrate | 1.38% |
| Silicone Gum⁶ | 0.5% |
| Fragrance | 5.5% |
| Complexed Beta Cyclodextrin | 3% |
| Total | 100 |

¹86% assay of anhydrous active, average particle size approximately 15 microns.
²Inventive Antibacterial Particle as made in Example 4
³DC 200 Fluid (50 centistoke) available from Dow Corning
⁴Tapioca Pure from Akzo Nobel
⁵Bentone 38 available from Elementis
⁶DC1503 (a mixture of dimethicone and dimethiconol) available from Dow Corning

Inventive Example 5 can be prepared by mixing a first portion of the dimethicone, isopropyl myristate and disteardimonium hectorite by lightly stirring followed by milling for at least 1 minute using a single head Silverson mill. The triethyl citrate is then added followed by at least five minutes of milling, followed by addition of the aluminum chlorohydrate, a second portion of the dimethicone, the complexed BCDs, the antibacterial particle, the tapioca material, dimethicone/dimethiconol and liquid fragrance material. Next, 25 g of the prepared composition is added to an appropriate spray canister, which is then sealed by an appropriate aerosol valve. Finally, 75 g of A46 propellant is added to the sealed container using a burette or other appropriate propellant filling equipment.

Throughout this specification, components referred to in the singular are to be understood as referring to both a single or plural of such component.

All percentages stated herein are by weight unless otherwise specified.

Every numerical range given throughout this specification will include every narrower numerical range that falls within such broader numerical range, as if such narrower numerical range were all expressly written herein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.1, 3.5 to 7.8, 5.5 to 10, etc.

Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An antibacterial particle comprising a cationically charged polymer and a salt.

2. The particle of claim 1, wherein the particle is anhydrous.

3. The particle of claim 1, wherein the cationically charged polymer is selected from the group consisting of primary and secondary amines.

4. The particle of claim 1, wherein the cationically charged polymer has a minimum molecular weight of 2000, and has at least about 30% of monomers with a cationic charge.

5. The particle of claim 1, wherein the salt is a water soluble, inorganic salt.

6. The particle of claim 1, wherein the salt has a hygroscopicity of less than about 40% at 80% humidity.

7. The particle of claim 1, wherein the cationically charged polymer is selected from the group consisting of polyvinyl amine, polyvinyle amine polyvinyl formide copolymer, polyquaterium polymers, polylysine, and combinations thereof.

8. The particle of claim 1, wherein the salt is selected from the group consisting of sodium chloride, potassium chloride, sodium potassium aluminum sulfate (alum), aluminum sulfate, aluminum chlorohydrate, aluminum zirconium salts, lithium chloride, calcium chloride, magnesium chloride, and combinations thereof.

9. The particle of claim 1, where the particle has a particle size of at most about 75 microns.

10. The particle of claim 1, wherein the particle has a particle size from about 5 microns to about 50 microns.

11. The particle of claim 1, wherein the particle is incorporated into an antiperspirant or deodorant composition.

12. The particle of claim 11, wherein the antiperspirant or deodorant composition is anhydrous.

13. The particle of claim 11, wherein the antiperspirant or deodorant composition is a stick, cream, or spray.

14. The particle of claim 11, wherein the antiperspirant or deodorant composition comprises less than about 5% volatile cyclic silicone.

15. The particle of claim 11, wherein the antiperspirant or deodorant composition is substantially free of aluminum.

16. A method of producing an antibacterial particle, comprising the steps of
a) providing a solution of a cationically charged polymer;
b) adding a salt in at least about 5 times in excess of the cationically charged polymer;
c) drying the polymer and salt solution to a friable particle;
d) grinding the particle to less than about 75 microns.

17. The method of claim 16, wherein the antibacterial particle is incorporated into an antiperspirant or deodorant composition.

18. An antiperspirant or deodorant product comprising an antibacterial particle comprising a cationically charged polymer and a salt, wherein the product contains less than about 5% volatile cyclic silicone.

19. The antiperspirant or deodorant product of claim 18, wherein the product is free from added volatile cyclic silicone.

20. The antiperspirant or deodorant product of claim 18, wherein the product is free of aluminum.
